(19) United States
(12) Patent Application Publication
(10) Pub. No.: US 2016/0303273 A1
(43) Pub. Date: Oct. 20, 2016

(54) DRESSING WITH ION-CARRYING COMPOSITION

(71) Applicant: 3M INNOVATIVE PROPERTIES COMPANY, St Paul, MN (US)

(72) Inventors: DAVID R. HOLM, HUDSON, WI (US); CARY A. KIPKE, AUSTIN, TX (US)

(73) Assignee: 3M INNOVATIVE PROPERTIES COMPANY

(21) Appl. No.: 15/183,858

(22) Filed: Jun. 28, 2016

Related U.S. Application Data
(63) Continuation of application No. 14/232,649, filed on Jan. 14, 2014, now abandoned.
(60) Provisional application No. 61/509,702, filed on Jul. 20, 2011.

(51) Int. Cl. A61L 15/44; A61F 13/02; A61L 15/18; A61F 13/00; A61L 15/22; A61L 15/42

(52) U.S. Cl. CPC A61L 15/44; A61L 15/225; A61L 15/425; A61L 15/18; A61F 13/00063; A61F 13/0203; A61F 13/0289; A61F 13/022; A61F 13/0216; A61L 2300/102

(57) ABSTRACT

A wound dressing comprising an open-cell foam substrate infused with an ion-containing composition in a water-dispersible carrier is provided. The composition includes citric acid or a salt thereof; salts of potassium, rubidium, and zinc ions; and a carrier comprising two or more polyol components, at least one polyol component being solid at 23 degrees C. A method of treating a wound with said wound dressing is also provided.

### CROSS REFERENCE TO RELATED APPLICATIONS

This application is a continuation of U.S. patent application Ser. No. 14/232,649, filed Jan. 14, 2014, which is a national stage filing under 35 U.S.C. 371 of PCT/US2012/047267, filed Jul. 19, 2012, which claims the benefit of U.S. Provisional Patent Application No. 61/509,702, filed Jul. 20, 2011, which is incorporated herein by reference in its entirety.

### BACKGROUND

The present application relates to wound dressings comprising compositions of aqueous oak bark extract and/or synthetic compositions containing the key active ingredients of oak bark extract and to the use of such dressings in the treatment of wounds of all types and, in particular, in the treatment of chronic skin ulcers.

Oak bark extract is described in U.S. Pat. No. 5,080,900 for use in the treatment of skin ulcers, particularly decubitus ulcers or bed sores. This material in a base of WHITFIELD'S ointment has also been sold under the trade name BENCELOK® for use in the treatment of minor skin irritations. The amount of oak bark extract in these materials was relatively low, however. For example, the BENCELOK® preparations have continued from 0.25 to 3% by weight of ash-derived components based upon the total weight of the preparation.

Synthetic compositions containing the key active ingredients of oak bark extract are used in wound dressings sold under the trade name TEGADERM MATRIX.

### SUMMARY

In general, the invention is directed to a wound dressing and a method of use thereof. The wound dressing is imbued with an ion-containing composition that promotes the healing of a variety of wounds including, for example, chronic skin ulcers. Advantageously, the dressing can release a bolus of ions after initial contact with an aqueous liquid (e.g., wound fluid). Furthermore, the dressing can continue to release an effective amount of ions for an extended period of time (e.g., up to at least 144 hours).

The composition with which the dressing is imbued comprises a carrier having two or more polyol components, at least one of the polyol components having a melting point higher than 23 degrees C. The composition of the carrier can be selected such that it confers one or more desirable properties on the wound dressing including relative rigidity and/or lubricity at room temperature, for example.

In one embodiment, the invention provides a wound dressing. The wound dressing can comprise an open-cell foam substrate having a perimeter and a composition infused therein. The composition can include citric acid or a salt thereof; salts of potassium, rubidium, and zinc ions; and a carrier comprising two or more polyol components, at least one of the polyol components being solid at 23 degrees C.

In any of the embodiments, the open-cell foam can comprise polyurethane foam carboxylated butadiene-styrene rubber based foams, polyester foams, and polyacrylate foams. In any of the above embodiments, the wound dressing further can comprise a backing layer contacting the substrate, wherein the backing layer has a first major surface and a second major surface, wherein the second major surface contacts the substrate. In any of the above embodiments, the composition further can comprise benzoic acid, a salt of benzoic acid, or a calcium salt. In any of the above embodiments, the carrier can comprise a first polyol component having an average formula weight of about 4000 daltons and a second polyol component having an average formula weight of less than or equal to about 400 daltons. In any of the above embodiments, one of the polyol components is selected from the group consisting of glycerol, propylene glycol, polyethylene glycol, polypropylene glycol and a block copolymer of polyethylene glycol and polypropylene glycol. In any of the above embodiments, the substrate can have a thickness of about 1 mm to about 20 mm. In any of the above embodiments, the pH range of the composition can be about 3.5 to about 7.0.

In any of the above embodiments, the composition can be substantially solid at 23 degrees C. and the wound dressing further can include a second composition. The second composition can comprise citric acid or a salt thereof; salts of potassium, rubidium, and zinc ions; and a carrier comprising two or more polyol components, at least one of the polyol components being solid at 23 degrees C. The second composition can be substantially liquid at 23 degrees C.

In another embodiment, the invention provides a method of treating a wound. The method can comprise providing a dressing comprising an open-cell foam substrate with a wound-facing major surface, the substrate infused with a composition comprising a plurality of inorganic ions and a mixture of two or more polyols; and contacting the dressing with a wound site. In any embodiment of the method, the composition can be substantially solid at 23 degrees C. and contacting the dressing with the wound site further can comprise contacting the dressing with the wound site under conditions sufficient to liquefy at least a portion of the composition to deliver an effective amount of the inorganic ions from the dressing into the wound site to promote wound healing.

The invention may provide a number of advantages. For example, the ions in the composition can facilitate biological processes that hasten the healing of a wound. In addition, the wound dressing can provide access for the ions to the wound over an extended period of time, thereby obviating the need for frequent dressing changes to maintain the delivery of effective amounts of the ions. Furthermore, the dressing can absorb wound exudate, thus providing a means for fluid management at a wound site and simultaneously maintaining a moist wound-healing environment.

Additional details of these and other embodiments are set forth in the accompanying drawings and the description below. Other features, objects and advantages will become apparent from the description and drawings, and from the claims.

### BRIEF DESCRIPTION OF DRAWINGS

FIG. 1 is an exploded perspective view of one embodiment of a wound dressing according to the present disclosure.

FIG. 2 is a plan view of the patient-facing side of the wound dressing of FIG. 1

FIG. 3 is a cross-sectional view of one embodiment of a wound dressing comprising a backing layer and an overcoat.

### DETAILED DESCRIPTION

The present disclosure is directed to a wound dressing that comprises an ion-containing active composition in a water-dispersible carrier. Ion-containing compositions from oak bark extract, and related synthetic compositions useful for the treatment of skin conditions such as wounds, are described in U.S. Pat. Nos. 6,149,947 and 7,014,870, which each is incorporated herein by reference in its entirety. The patents disclose the incorporation of the active ingredients into a pharmaceutically-acceptable carrier, such as Whitfield's ointment.

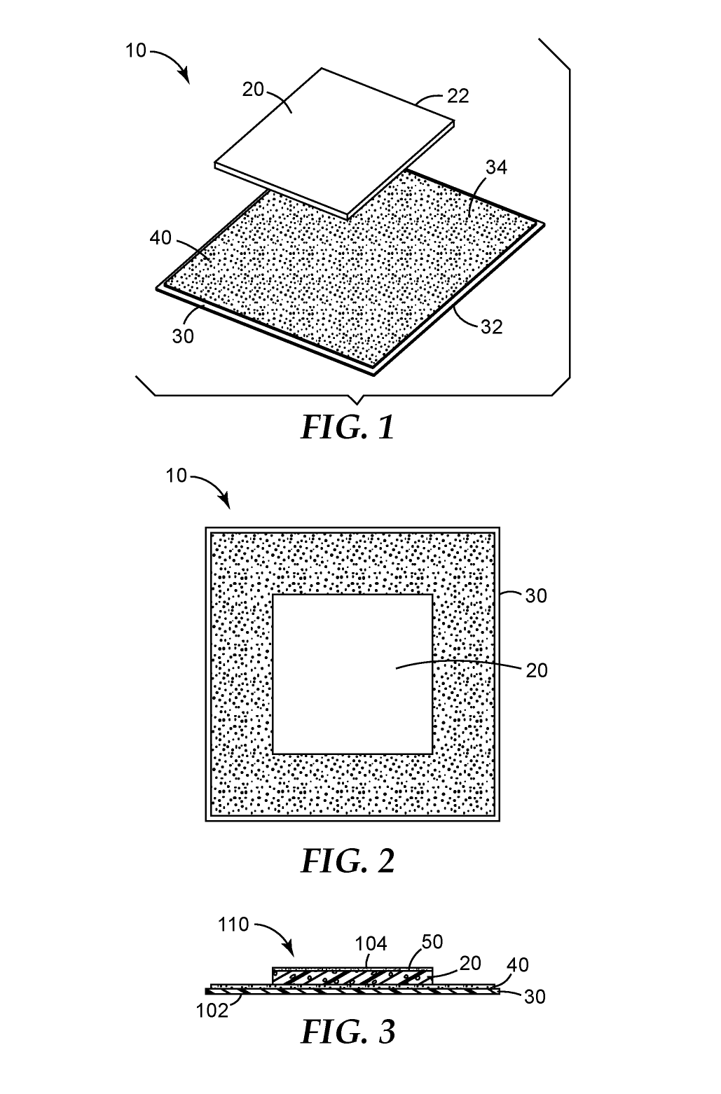

FIG. 1 is a partially-exploded perspective view of a wound dressing 10 according to the present disclosure. The wound dressing 10 comprises a coated substrate 20 that is infused with an ion-containing active composition in a water-dispersible carrier. The coated substrate 20 comprises open-cell foam. The coated substrate 20 can have a thickness from about 1 mm to about 20 mm. An exemplary open-cell foam is a polyurethane foam, such as that used in 3M TEGADERM FOAM (NON-ADHESIVE) dressing available from 3M Company (St. Paul, Minn.). Other exemplary foams include open-cell foams comprising carboxylated butadiene-styrene rubber based foams, polyester foams, and polyacrylate foams. The coated substrate 20 further comprises a perimeter 22.

The wound dressing 10 may further comprise an optional backing layer 30. The backing layer 30 comprises a first major surface 32 and a second major surface 34. The second major surface 34 contacts the coated substrate 20. Preferably, the backing material 30 comprises a substantially planar material. The backing layer 30 may be configured in one of a variety of thicknesses (e.g., about 10 microns thick to about 5000 microns thick). In some embodiments, the backing layer 30 can comprise a substantially moisture-impermeable material such as a polymeric film (e.g., polyurethane film, polyolefin film, or polyester film), for example. In some embodiments, the backing layer 30 can comprise moisture-absorbing material (e.g., foams or fibrous materials. In some embodiments, the backing layer may comprise a porous film. In some embodiments (not shown), the backing layer may comprise a laminate of a plurality of moisture-absorbing layers, moisture-impermeable layers, or combinations thereof.

In some embodiments, the backing layer 30 can be dimensioned to be at least co-extensive with the perimeter 22 of the coated substrate 20. In some embodiments, the backing layer 30 can be dimensioned such that a part of the backing layer 30 extends outside the perimeter 22 of the coated substrate 30, as shown in FIG. 2.

Optionally, a portion of the second major surface 34 can further comprise an adhesive layer 40. The adhesive layer 40 can function to adhere the backing layer 30 to the coated substrate 20 and/or to adhere the backing layer 30 to a patient (e.g., a patient's skin). The adhesive layer 40 can comprise any suitable adhesive (e.g., a pressure-sensitive adhesive) known in the art for the aforementioned purposes. The adhesive layer 40 can be applied to the backing layer 30 by any suitable process known in the art including, for example, knife-coating, spray coating, and kiss-coating.

Wound dressings of the present disclosure comprise an ion-containing composition in a water-dispersible carrier. The composition comprises salts of potassium, rubidium, and zinc ions. Optionally, the composition further can comprise a salt of calcium ions. Salts of the potassium, rubidium, zinc, and/or calcium ions may include chloride anions, for example. The composition further comprises citric acid or a salt thereof (e.g., potassium citrate). In any embodiment, the composition optionally further can comprise benzoic acid or a salt thereof. The composition can have a pH of about 3.5 to about 7.0.

The water-dispersible carrier comprises two or more polyol components. The term “polyol”, as used herein, refers to an alcohol having a plurality of hydroxyl groups. Nonlimiting examples of suitable polyols include glycerol, propylene glycol, polyethylene glycol, polypropylene glycol and block copolymers of polyethylene glycol and polypropylene glycol. At least one of the two or more polyol components in the water-dispersible carrier includes a polyol component (e.g., a polyethylene glycol component with an average molecular weight of about 4000-6000 daltons) that is a solid at 23 degrees C. In some embodiments, at least one of the two or more polyol components in the water-dispersible carrier includes a polyol component (e.g., glycerol and/or a polyethylene glycol component with an average molecular weight of about 400-700 daltons) that is a liquid at room temperature.

Advantageously, an aqueous mixture of the ions; with at least two polyol components that are liquid and solid, respectively, at 23 degrees C.; can be used to produce a composition with a pre-selected melting point. Table 1 shows non-limiting exemplary ion-containing compositions according to the present disclosure. Accordingly, the wound dressing can be coated with a composition that is in relatively-viscous waxy state at room temperature, but readily melts to a relatively low-viscosity liquid at normal body temperature (ca. 37 degrees C.).

TABLE 1
Exemplary ion-containing compositions in a carrier. All values are reported
as the weight percent (wt %) of each component in the final composition.

| | | | | | | | | | | | Physical |
|---|---|---|---|---|---|---|---|---|---|---|---|
| | PEG | PEG | | Citric | Potassium | Benzoic | Rubidium | Zinc | Calcium | | State |
| | 400 | 4000 | Glycerol | Acid | Citrate | Acid | Chloride | Chloride | Chloride | Water | (23° C.) |
| Mixture A | 32.4 | 40.0 | — | 2.5 | 2.0 | 0.1 | 0.1 | 0* | 0 | 22.9 | Solid |
| Mixture B | 25.8 | 31.9 | 14.7 | 2.5 | 2.0 | 0.1 | 0.1 | 0 | 0 | 22.9 | Solid |
| Mixture C | 20.9 | 25.8 | 25.8 | 2.5 | 2.0 | 0.1 | 0.1 | 0 | 0 | 22.9 | Solid |
| Mixture D | 15.4 | 19.0 | 38.0 | 2.5 | 2.0 | 0.1 | 0.1 | 0 | 0 | 22.9 | Liquid |
| Mixture E | 32.3 | 20.1 | 20.1 | 2.5 | 2.0 | 0.1 | 0.1 | 0 | 0 | 22.9 | Liquid |
| Mixture F | 25.3 | 15.7 | 31.4 | 2.5 | 2.0 | 0.1 | 0.1 | 0 | 0 | 22.9 | Liquid |
| Mixture G | 17.6 | 11.0 | 43.8 | 2.5 | 2.0 | 0.1 | 0.1 | 0 | 0 | 22.9 | Liquid |
| Mixture H | 37.6 | 11.6 | 23.3 | 2.5 | 2.0 | 0.1 | 0.1 | 0 | 0 | 22.9 | Liquid |
| Mixture I | 32.4 | 10.0 | 20.0 | 2.5 | 2.0 | 0.1 | 0.1 | 0 | 0 | 22.9 | Liquid |
| Mixture J | 24.2 | 24.2 | 24.2 | 2.5 | 2.0 | 0.1 | 0.1 | 0 | 0 | 22.9 | Liquid |
| Mixture K | 20.7 | 20.7 | 31.0 | 2.5 | 2.0 | 0.1 | 0.1 | 0 | 0 | 22.9 | Liquid |
| Mixture L | 18.1 | 18.1 | 36.2 | 2.5 | 2.0 | 0.1 | 0.1 | 0 | 0 | 22.9 | Liquid |

*Note: all values are rounded to the nearest 0.1%. A “—” means that the component was not present in the mixture. A “0” means the component was present at a concentration less than 0.1 wt %.

Wound dressings of the present disclosure can be made by a variety of processes. In some embodiments, it may be advantageous to produce a wound dressing comprising a composition that exists substantially as a liquid emulsion at room temperature, as described in Examples 4-10 herein. Dressings comprising a substantially liquid composition at room temperature may provide rapid deployment of the ions therein to the wound site. In contrast, in some embodiments, it may be advantageous to produce a wound dressing having a composition that exists substantially as a solid emulsion at room temperature. Dressings comprising a substantially solid composition at room temperature advantageously may provide deployment of the ions to the wound site over an extended period of time as the composition more-slowly liquefies and mixes with the wound fluid.

In some embodiments, the wound dressing may comprise two coatings—a first coating having a composition that has a first melting point (e.g., a melting point greater than 23 degrees C.) and a second coating having a composition that has a second melting point (e.g., a melting point less than or equal to 23 degrees C.). In some embodiments, a second composition having a melting point less than or equal to 23 degrees C. may be over-coated onto a dressing comprising a first composition having a melting point greater than 23 degrees C., as described below. In these embodiments, the second composition provides initial rapid deployment of a bolus of ions when the dressing contacts the wound (e.g., via the liquid overcoat) while the first composition also provides delivery of the ions to the wound for an extended period of time. In some embodiments, both the first and second compositions may have a melting point greater than 23 degrees C. and the combination of coatings may provide the desired release profile for the ions in the composition. FIG. 3 shows a side view of one embodiment of a wound dressing of the present disclosure with an overcoat. The dressing 110 has two major surfaces, an external surface 102 and a wound-facing surface 104. The dressing 110 comprises a coated substrate 20 comprising a composition that is substantially solid at 23 degrees C. Coated on at least a portion of the wound-facing surface 104 of the dressing 110 is an overcoat 50.

The composition can be applied to the substrate using a variety of processes that are known in the art including, for example, dip coating, spray coating, curtain coating, knife coating, and kiss coating. Preferably, the composition is maintained in a well-mixed, liquid state during the coating process. Thus, in some embodiments wherein the composition forms a solid at ambient temperature, it may be desirable to heat the composition before and, optionally, during the coating process to maintain a homogeneous mixture. Maintaining the composition in a liquid state during the coating process further will facilitate penetration of the composition into the interior regions of the substrate and, in some embodiments (e.g., when relatively low-viscosity mixtures are coated onto relatively thin substrates), through the entire thickness of the substrate.

In some embodiments, penetration of the composition into and/or through the substrate can be facilitated by expelling a portion of the air or gas present in one or more cells of the open-cell foam substrate. This can be performed by compressing the substrate (e.g., between a roller and a relatively noncompressible surface) immediately before and/or during contact between the substrate and a liquid coating mixture comprising the composition. Thus, as the foam substantially regains its original shape, the cells are filled or partially filled with the coating mixture.

In any embodiment, the coated substrate can be held in an environment (e.g., ambient temperature and/or humidity) for a period of time (e.g., up to several days). This can allow the composition to revert to a substantially solid or semi-solid state, for example.

In some embodiments (e.g., wherein the composition exists in a substantially solid or semi-solid form in the coated substrate at 23 degrees C.), an overcoat may be applied to the wound dressing. The overcoat may, for example, comprise a second ion-containing composition with a carrier that exists in a substantially liquid form at 23 degrees C., as described above.

The present disclosure provides a method of treating a wound. The method comprises providing a dressing according to any of the above embodiments. In some embodiments, the dressing comprises an open-cell foam substrate with a wound-facing major surface, the substrate infused with a composition comprising a plurality of inorganic ions and a carrier comprising a mixture of two or more polyols. The method further comprises contacting the dressing with a wound site. In some embodiments, the composition infused in the wound dressing is substantially solid at 23 degrees C. In these embodiments, the dressing is contacted with the wound site under conditions sufficient to liquefy at least a portion of the solid composition to deliver an effective amount of the inorganic ions from the dressing into the wound site to promote wound healing. Advantageously, the foam substrate acts as an insulator to maintain the temperature of the composition proximate the wound in a liquid state.

### Embodiments

Embodiment 1 is a wound dressing, comprising:
an open-cell foam substrate having a perimeter; and
a composition infused therein, the composition including;
   citric acid or a salt thereof;
   salts of potassium, rubidium, and zinc ions; and
a carrier comprising two or more polyol components, at least one of the polyol components being solid at 23 degrees C.

Embodiment 2 is the wound dressing of claim 1, wherein the open-cell foam comprises polyurethane foam carboxylated butadiene-styrene rubber based foams, polyester foams, and polyacrylate foams.

Embodiment 3 is the wound dressing of any one of the preceding claims, further comprising a backing layer having a first major surface and a second major surface, wherein the second major surface contacts the substrate.

Embodiment 4 is the wound dressing of claim 3, wherein a portion of the second major surface further comprises an adhesive layer.

Embodiment 5 is the wound dressing of claim 3 or claim 4, wherein the backing layer further comprises a part that extends outside the perimeter of the substrate.

Embodiment 6 is the wound dressing of any one of the preceding claims, wherein the composition further comprises benzoic acid, a salt of benzoic acid, or a calcium salt.

Embodiment 7 is the wound dressing of any one of the preceding claims, wherein the carrier comprises a first polyol component having an average formula weight of about 4000-6000 daltons and a second polyol component having an average formula weight of less than or equal to about 600 daltons.

Embodiment 8 is the wound dressing of any one of the preceding claims, wherein one of the polyol components is selected from the group consisting of glycerol, propylene glycol, polyethylene glycol, polypropylene glycol and a block copolymer of polyethylene glycol and polypropylene glycol.

Embodiment 9 is the wound dressing of any one of the preceding claims, wherein the substrate has a thickness of about 1 mm to about 20 mm.

Embodiment 10 is the wound dressing of any one of the preceding claims, wherein the pH range of the composition is about 3.5 to about 7.0.

Embodiment 11 is the wound dressing of any one of the preceding claims;
wherein the composition is substantially solid at 23 degrees C.;
wherein the wound dressing further includes a second composition;
wherein the second composition comprises citric acid or a salt thereof; salts of potassium, rubidium, and zinc ions; and a carrier comprising two or more polyol components, at least one of the polyol components being solid at 23 degrees C.;
wherein the second composition is substantially liquid at 23 degrees C.

Embodiment 12 is a method of treating a wound, comprising:
providing a dressing comprising an open-cell foam substrate with a wound-facing major surface, the substrate infused with a composition comprising a plurality of inorganic ions and a carrier comprising a mixture of two or more polyols; and
contacting the dressing with a wound site.

Embodiment 13 is the method of claim 12, wherein the composition is substantially solid at 23 degrees C. and wherein contacting the dressing with the wound site further comprises contacting the dressing with the wound site under conditions sufficient to liquefy at least a portion of the composition to deliver an effective amount of the inorganic ions from the dressing into the wound site to promote wound healing.

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is distilled water, and all molecular weights are weight average molecular weight.

### EXAMPLES

Materials utilized in the preparation of the examples are shown in Table 2.

TABLE 2

| Component | Source | Description |
|---|---|---|
| Citric Acid | VWR | Anhydrous |
| Potassium Citrate | J. T. Baker | Monohydrate, USP |
| Rubidium Chloride | Alfa Aesar | |
| Zinc Chloride | EMD | USP, Ph Eur |
| Calcium Chloride | Sigma | Dihydrate |
| Water | BDH | Purified Water, USP |
| PEG 400 | EMD | Average molecular weight |
| | | of 380-420 |
| PEG 4000 | EMD | Average molecular weight |
| | | of 3500 |
| Benzoic Acid | EMD | Ph Eur, USP |
| Glycerol | J. T. Baker | |
| Non-woven | Ahlstrom | 70% rayon/30% PET, |
| | (Green Bay, WI) | 40 g/m² |
| Foam | 3M Tegaderm ™ | Polyurethane |
| | Foam (Non-adhesive) | |
| Penicillin- | Sigma-Aldrich | Part Number P0781 |
| Streptomycin | | |
| Water | | De-ionized and filtered |

Aqueous Salt Solution

An aqueous salt solution was prepared by mixing citric acid (55.4 g), potassium citrate (42.8 g), rubidium chloride (1.7 g), zinc chloride (0.004 g), and calcium chloride (0.01 g) in sterile water (473.2 g). This aqueous salt solution was used in the formulations described in Examples 1-12.

Test Methods

Viscosity

A Brookfield viscometer (Model LTV, Brookfield Engineering Laboratories, Inc. Middleboro, Mass.) equipped with a LV4 spindle was used to conduct viscosity experiments. The viscosity experiments were conducted at ambient temperature with a spindle rotation of 6 rpm.

Potassium Ion Concentration

Standard potassium solutions were utilized to create a standard curve. Potassium concentration was measured with a MA 235 pH/ion analyzer (Mettler-Toledo, Columbus, Ohio) equipped with a potassium Ionplus®, Sure-Flow® plastic membrane ion selective electrode (Thermo Fisher Scientific, Beverly, Mass.).

### Example 1

### Ion-Containing Formulation

A 500 mL flask equipped with a temperature control probe to control a heating mantle was charged with PEG 400 (73.67 g). The solution was heated to 65 C. with mechanical stirring and PEG 4,000 (90.95 g) was slowly added to the solution. After the PEG 4,000 had dissolved, benzoic acid was added (0.22 g) and stirred for approximately 5 minutes. Aqueous Salt Solution (52 mL) was then added to the solution and stirred for 5 minutes. The resulting mixture was cooled to room temperature to form a viscous, waxy emulsion (83,000 cps).

### Examples 2-12

### Ion-Containing Formulations

Examples 2-12 were prepared in a similar manor as Example 1. In all Examples, 0.22 g benzoic acid and 52 mL Aqueous Salt Solution were utilized. Compositions for all Examples are shown in Table 3.

TABLE 3
Example Formulations

| Exam- | PEG 400 | Glycerol | PEG 4000 | Viscosity | |
|---|---|---|---|---|---|
| ple | (g) | (g) | (g) | (cps) | Observation |
| 1 | 73.67 | 0 | 90.95 | 83,000 | emulsion |
| 2 | 58.74 | 33.36 | 72.52 | 12,500 | emulsion |
| 3 | 47.45 | 58.58 | 58.58 | 25,000 | emulsion |
| 4 | 35.00 | 86.42 | 43.21 | 300 | phase-separated |
| 5 | 73.42 | 45.60 | 45.60 | 1,900 | phase-separated |
| 6 | 57.49 | 71.42 | 35.71 | 300 | phase-separated |
| 7 | 40.10 | 99.62 | 24.90 | 100 | phase-separated |
| 8 | 85.35 | 52.85 | 26.42 | 100 | phase-separated |
| 9 | 73.54 | 45.54 | 22.77 | 200 | phase-separated |
| 10 | 54.87 | 54.87 | 54.87 | 1,900 | phase-separated |
| 11 | 47.03 | 70.55 | 47.03 | —[a] | emulsion |
| 12 | 41.16 | 82.31 | 41.16 | 2,000 | emulsion |

[a] Not Measured

### Examples 13-22

### Foam Dressings Comprising Ion-Containing Formulations.

A non-woven material was placed on a plastic liner in an aluminum tray. The Ion-containing formulations (described above) were heated to 60° C. and allowed to absorb into the non-woven. For each example, a 2.54 cm square (ca. 0.8 cm-thick) piece of foam was coated with the respective ion-containing composition. The foam was the same composition and approximate thickness as the foam that can be obtained by separating (e.g., by excising) the foam in a 3M TEGADERM Foam Dressing (Non-Adhesive; part number 90601; available from 3M Company, St. Paul, Minn.) from the backing layer. Each square piece was placed on the saturated non-woven sample and a rubber roller was used to provide pressure on the foam sample. The tool was rolled over the entire surface of the foam for approximately 10-15 seconds to ensure that a sufficient amount of the ion-containing formulation absorbed into the foam. The resulting coated foam sample was covered with a plastic liner.

After a 2 -3 day exposure to ambient conditions the coated foam samples were weighed and a coat weight was determined. The coated samples were placed in foil packages and subjected to gamma irradiation at a dose of 35 kGy. The coat weight (grams per foam square) for each coated foam sample is shown in Table 4.

TABLE 4
Sample Coat Weights

| | Ion-containing Formulation | Coat Weight |
|---|---|---|
| Example # | (Example #) | (g) |
| 13 | 3 | 3.21 |
| 14 | 3 | 3.18 |
| 15 | 3 | 3.59 |
| 16 | 3 | 3.18 |
| 17 | 3 | 6.31 |
| 18 | 4 | 3.68 |
| 19 | 4 | 3.02 |
| 20 | 4 | 3.78 |
| 21 | 4 | 3.10 |
| 22 | 4 | 4.23 |

Ion Diffusion Kinetics Experiments—Flow System

The kinetic ion release from the foam samples was conducted using an AKTA Fast Performance Liquid Chromatography (FPLC™) system (Amersham Pharmacia Biotech, Uppsala, Sweden) equipped with sample holders fabricated by Classic Manufacturing Inc. (Oakdale, Minn.). The sample holders were designed to accommodate a 47 mm diameter foam sample and provide channels for liquid flow through the foam.

Seven samples of coated foam; corresponding to Example numbers 13, 15, 17, 18, 20, 21, and 22; were utilized to measure ion release from a 0.8cm-thick foam. A 47 mm diameter disk was die-cut from the 4″×4″ foam samples and placed in the sample holder. An O-ring was placed on top of the foam disc and the sample holder top was screwed to the sample holder bottom until compression with the o-ring was established. The cartridge/foam assembly was connected to the AKTA FPLC. Each sample was hydrated with sterile water flowing at 1 mL/min until all air was removed from the cartridge. Water was passed through the sample holder at a flow rate of approximately 0.2 mL/minute. A 2 mL aliquot was collected initially, at approximately 16 hours, and at approximately 5.5 hour increments thereafter. The potassium ion concentration was determined in these aliquots. The results are presented in Tables 5-11. The results indicate the presence of at least 34 ppm potassium ion in every sample up to 94 hours of contact with the disks.

TABLE 5
Potassium ion concentration in liquid samples (Example 13). Time is reported as the total numberof hours since the cartridge was
initially filled with sterile water. All concentrations of potassium ion are reported in parts per million (ppm).

| | Time (hr) | | | | | | | | | | | | | | | | |
|---|---|---|---|---|---|---|---|---|---|---|---|---|---|---|---|---|---|
| | 4.75 | 20.9 | 26.5 | 32.3 | 38.2 | 44 | 49.8 | 55.7 | 61.5 | 67.3 | 73.2 | 79 | 84.8 | 90.7 | 96.5 | 102.3 | 108.2 |
| Conc. | 198 | 226 | 182 | 154 | 128 | 111 | 100 | 81 | 74 | 62 | 58 | 52 | 44 | 37 | 32 | 29 | 25 |
| [K⁺] | | | | | | | | | | | | | | | | | |

TABLE 6
Potassium ion concentration in liquid samples (Example 15). Time is reported as the total number of hours since the cartridge was
initially filled with sterile water. All concentrations of potassium ion are reported in parts per million (ppm).

| | Time (hr) | | | | | | | | | | | | | | | | |
|---|---|---|---|---|---|---|---|---|---|---|---|---|---|---|---|---|---|
| | 1 | 17.2 | 22.3 | 28.2 | 34 | 39.8 | 45.7 | 51.5 | 57.3 | 63.2 | 69 | 74.8 | 80.7 | 86.5 | 92.3 | 98.2 | 104 |
| Conc. | 169 | 409 | 392 | 316 | 257 | 232 | 205 | 168 | 161 | 135 | 119 | 106 | 95 | 85 | 74 | 73 | 47 |
| [K⁺] | | | | | | | | | | | | | | | | | |

TABLE 7
Potassium ion concentration in liquid samples (Example 17). Time is reported as the total number of hours since the cartridge was
initially filled with sterile water. All concentrations of potassium ion are reported in parts per million (ppm).

| | Time (hr) | | | | | | | | | | | | | | | | |
|---|---|---|---|---|---|---|---|---|---|---|---|---|---|---|---|---|---|
| | 5.5 | 21.8 | 27.3 | 33.2 | 39 | 44.8 | 50.7 | 56.5 | 62.3 | 68.2 | 74 | 79.8 | 85.7 | 91.5 | 97.3 | 103.2 | 109 |
| Conc. | 416 | 502 | 408 | 331 | 273 | 241 | 213 | 181 | 112 | 136 | 128 | 107 | 93 | 82 | 68 | 60 | 51 |
| [K⁺] | | | | | | | | | | | | | | | | | |

TABLE 8
Potassium ion concentration in liquid samples (Example 18). Time is reported as the total number of hours since the
cartridge was initially filled with sterile water. All concentrations of potassium ion are reported in parts per million (ppm).

| | Time (hr) | | | | | | | | | | | | | | | | |
|---|---|---|---|---|---|---|---|---|---|---|---|---|---|---|---|---|---|
| | 4 | 20.2 | 25.7 | 31.5 | 37.3 | 43.2 | 49 | 54.8 | 60.7 | 66.5 | 72.3 | 78.2 | 84 | 89.8 | 95.7 | 101.5 | 107.3 |
| Conc. | 221 | 306 | 256 | 208 | 175 | 152 | 131 | 115 | 97 | 84 | 80 | 67 | 62 | 51 | 44 | 37 | 32 |
| [K⁺] | | | | | | | | | | | | | | | | | |

TABLE 9
Potassium ion concentration in liquid samples (Example 20). Time is reported as
the total number of hours since the cartridge was initially filled with sterile water.
All concentrations of potassium ion are reported in parts per million (ppm).

| | | | | | | | | | | | | | | | |
|---|---|---|---|---|---|---|---|---|---|---|---|---|---|---|---|
| Time | 2.5 | 18.7 | 24 | 29.8 | 35.7 | 41.5 | 47.3 | 53.2 | 59 | 64.8 | 70.7 | 76.5 | 82.3 | 88.2 | 94 |
| (hr) | | | | | | | | | | | | | | | |
| Conc. | 141 | 205 | 175 | 142 | 119 | 103 | 91 | 81 | 68 | 57 | 54 | 49 | 44 | 38 | 34 |
| [K⁺] | | | | | | | | | | | | | | | |

TABLE 10
Potassium ion concentration in liquid samples (Example 21). Time is reported as the total number of hours since the
cartridge was initially filled with sterile water. All concentrations of potassium ion are reported in parts per million (ppm).

| | Time (hr) | | | | | | | | | | | | | | | | |
|---|---|---|---|---|---|---|---|---|---|---|---|---|---|---|---|---|---|
| | 1.8 | 17.9 | 23.2 | 29 | 34.8 | 40.7 | 46.5 | 52.3 | 58.2 | 64 | 69.8 | 75.7 | 81.5 | 87.3 | 93.2 | 99 | 104.8 |
| Conc. | 146 | 354 | 339 | 273 | 221 | 201 | 177 | 145 | 139 | 116 | 102 | 92 | 82 | 73 | 63 | 62 | 40 |
| [K⁺] | | | | | | | | | | | | | | | | | |

TABLE 11
Potassium ion concentration in liquid samples (Example 22). Time is reported as the total number of hours since the
cartridge was initially filled with sterile water. All concentrations of potassium ion are reported in parts per million (ppm).

| | Time (hr) | | | | | | | | | | | | | | | | |
|---|---|---|---|---|---|---|---|---|---|---|---|---|---|---|---|---|---|
| | 3.2 | 19.4 | 24.8 | 30.7 | 36.5 | 42.3 | 48.2 | 54 | 59.8 | 65.7 | 71.5 | 77.3 | 83.2 | 89 | 94.8 | 100.7 | 106.5 |
| Conc. | 148 | 217 | 184 | 149 | 124 | 106 | 93 | 84 | 70 | 58 | 55 | 50 | 45 | 38 | 34 | 30 | 26 |
| [K⁺] | | | | | | | | | | | | | | | | | |

Ion Diffusion Kinetics Experiments—Static System

The kinetic ion release from the foam samples was conducted using a 6-well microtiter plate (BD Falcon Multiwell Flat-Bottom Plates with Lids, Sterile; part number 62406-155; VWR, Radnor, Pa.). Circular disks of foam samples from Example numbers 15, 16, and 17 were prepared using a 31.75 mm-diameter punch die. A soak solution containing 1 mL of Penicillin-Streptomycin per 100 mL deionized water was prepared (final concentrations of the antibiotics in the soak solution were 100 U/mL penicillin and 0.1 mg/mL streptomycin, respectively).

Duplicate foam samples of each dressing composition were individually placed into separate wells of the 6-well plates and each foam sample was contacted with 3 milliliters of the soak solution. An additional volume (2 milliliters) of the soak solution was immediately added to each microwell to start the experiment and the microwell plates were placed in a humidified incubator set at 37° C. Two-milliliters of liquid sample were removed from each microwell for potassium ion analysis at each of the time points listed in Table 4. The removed liquid was immediately replaced with two-milliliters of soak solution. Thus, each disk that was subjected to ten different contact times was contacted with a total of twenty milliliters of the soak solution. Even after nine separate contacts with 2 milliliters of soak solution, the foam dressings of Examples 15 and 16 released over 30 ppm of potassium ion into the soak solution. In contrast, after only six separate contacts with 2 milliliters of soak solution, the TEGADERM MATRIX dressings released less than 20 ppm of potassium ion into the soak solution.

As a comparison, two lots of commercially-available 3M TEGADERM MATRIX dressings (3M Company, St. Paul, Minn.) were subjected to the same ion diffusion kinetic experiments. The dressings were cut into 31.75-mm disks and placed into separate wells of the microtiter plates, as described above.

Potassium ion was quantified in the liquid samples using a potassium ion probe, as described above. The probe was calibrated using a standards ranging from 0 mM to 100 mM potassium ion. The results are shown in Table 12.

TABLE 12
Potassium ion concentration in liquid samples. All data represent the
average of the duplicate disk samples for each construction.

| Contact | | | | TEGADERM | TEGADERM |
|---|---|---|---|---|---|
| Time | Exam- | Exam- | Example | MATRIX | MATRIX |
| (hrs) | ple 15 | ple 16 | 17 | (Lot 1) | (Lot 2) |
| 0.5 | 1,276.3 | 813.8 | 874.3 | 512.7 | 411.6 |
| 0.5 | 875.6 | 571.0 | 527.5 | 298.9 | 220.0 |
| 1 | 740.6 | 467.3 | 376.8 | 186.6 | 154.1 |
| 4 | 403.0 | 295.8 | 221.0 | 108.5 | 84.7 |
| 19 | 283.2 | 216.1 | 167.8 | 63.8 | 52.3 |
| 6.5 | 165.8 | 131.9 | 117.9 | 32.6 | 25.5 |
| 17.5 | 125.2 | 104.2 | 94.7 | 19.5 | 15.7 |
| 8 | 74.3 | 71.1 | 66.5 | 9.1 | 7.3 |
| 18.25 | 48.3 | 53.4 | ND | 4.5 | 3.6 |
| 73.25 | 34.1 | 41.0 | ND | 2.4 | 2.0 |

ND = Not detected

A number of embodiments of a wound dressing have been described. For example, various compositions, each composition comprising inorganic ions and having a characteristic melting point, are described. In particular, the dressings release an effective amount of the inorganic ions into an aqueous environment (e.g., wound fluid) for an extended period of time.

Nevertheless, various modifications may be made without departing from the spirit and scope of the invention. For example, one or more features described herein may be used with or without other described features. Moreover, several features described herein may be used in a wound dressing that can remain in contact with a wound site for an extended period of time. These and other embodiments are within the scope of the following claims.

1. A method of making a wound dressing, the method comprising compressing and then decompressing an open-cell foam substrate while the substrate contacts a liquid coating composition, wherein the liquid coating composition comprises two or more polyol components, wherein at least one of the polyol components has a melting point above 23 degrees C.

2. The method of claim 1, wherein the open-cell foam comprises polyurethane foam carboxylated butadiene-styrene rubber based foams, polyester foams, and polyacrylate foams.

3. The method of claim 1 wherein, prior to contacting the substrate with the liquid coating composition, the method further comprises heating the coating composition above 23 degrees C.

4. The method of claim 3, wherein heating the coating composition above 23 degrees C. comprises heating the coating composition to about 60 degrees C. or above.

5. The method of claim 4, wherein heating the coating composition above 23 degrees C. comprises heating the coating composition to about 60-65 degrees C.

6. The method of claim 1, further comprising a step of adhering the substrate to a backing layer.

7. The method of claim 6, wherein the backing layer comprises a moisture-absorbing material.

8. The method of claim 1, wherein the backing layer comprises a moisture-impermeable material.

9. The method of claim 1, wherein the backing layer comprises a laminate of a plurality of moisture-absorbing layers, moisture-impermeable layers, or combinations thereof.

10. The method of claim 1, wherein the coating composition further comprises:
citric acid or a salt thereof; and
salts of potassium, rubidium, and zinc ions.

11. The method of claim 1, wherein the coating composition further comprises benzoic acid, a salt of benzoic acid, or a calcium salt.

12. The method of claim 1, wherein the two or more polyol components comprise a first polyol component having an average formula weight of about 4000-6000 daltons and a second polyol component having an average formula weight of less than or equal to about 600 daltons.

13. The method of claim 12, one of the two or more polyol components is selected from the group consisting of glycerol, propylene glycol, polyethylene glycol, polypropylene glycol and a block copolymer of polyethylene glycol and polypropylene glycol.

14. The method of claim 1, wherein the substrate has a thickness of about 1 mm to about 20 mm.

15. The method of claim 1, wherein the pH range of the coating composition is about 3.5 to about 7.0.
